(19) United States
(12) Patent Application Publication
(10) Pub. No.: US 2019/0001332 A1
(43) Pub. Date: Jan. 3, 2019

(54) DISPOSABLE INDEPENDENT 3-D STRUCTURE DEPENED SEQUENTIAL CAPILLARY LATERAL FLOW DEVICE FOR ANALYTE DETERMINATION

(71) Applicant: Gene Bio Application Ltd., Yavne (IL)

(72) Inventors: Yitzhak Ben Asouli, Kfar Hanagid (IL); Farhat Osman, Sachnin (IL)

(73) Assignee: Gene Bio Application Ltd., Yavne (IL)

(21) Appl. No.: 16/063,372

(22) PCT Filed: Dec. 19, 2016
(86) PCT No.: PCT/IL2016/051355

§ 371 Date: Jun. 18, 2018

Related U.S. Application Data
(60) Provisional application No. 62/270,026, filed on Dec. 20, 2015.

(51) Int. Cl. B01L 3/00; G01N 33/558; G01N 33/53; B81B 1/00; B01L 9/00

(52) U.S. Cl. CPC B01L 3/502746; G01N 33/558; G01N 33/5302; B01L 3/502769; B01L 9/527; B01L 3/502715; B81B 1/00

(57) ABSTRACT

The present invention provides a disposable 3-D structured depended sequential lateral flow capillary device comprising: a housing; a 3-D structured capillary flow matrix; and (iii) an absorption portion, and use thereof in a method for determining the presence of an analyte in a sample.

### FIELD OF THE INVENTION

The present invention relates to the field of analytes detection. More particularly, the present invention relates to a simple to use, disposable sequential lateral flow capillary device which does not require electric power, and methods of using same for detecting analytes and performing binding assays.

### BACKGROUND OF THE INVENTION

Specific binding assays are essential for a variety of clinical and research applications (see e.g. WO 2005/031355). Specific binding assays involve the detection and preferably quantitative determination of a specific analyte in a sample, usually by the binding of the analyte to a specific receptor, e.g. an antibody. Examples of specific binding assays are immunological assays involving reactions between antibodies and antigens; hybridization reactions of DNA and RNA, etc. Specific binding assays may be practiced according to a variety of methods known to the art, such as competitive binding assays, “direct” and “indirect” sandwich assays (U.S. Pat. No. 4,861,711; U.S. Pat. No. 5,120,643), etc.

Since the complex formed of by a specific binding reaction is usually not directly observable, different labeling techniques were developed. Known labels include radiolabels, chromophores and fluorophores, and enzymes, the presence of which may be detected by radiation detectors, spectrophotometers or the naked eye.

Lateral flow capillary devices are well known in the fields of analysis and detection, and are often used for quick and simple implementation of specific binding assay of analyte in a liquid sample. Such devices usually employ the placing a sample into an area of the device comprising a soluble labeled reagent configured to bind to the desired analyte; then, the labeled analyte migrates through a capillary flow matrix towards a drain area (having high absorbent properties); during this migration, the labeled analyte reaches a pre-defined reaction zone which comprises anti-analytes that bind and anchor the labeled analyte; the bound labeled analyte accumulate at this reaction zone thus becoming visible (usually through a window in the device above this reaction zone). Such lateral flow capillary devices are useful and simple to use, and are relatively cheap to produce. However, they have several disadvantages, such as: losing large amount of the sample (and desired analyte); and inadequacy for multistep reactions, which forces the user to add liquids serially.

Since multistep binding assays are known to be more sensitive and accurate than single step binding assays, other lateral flow capillary devices were developed: U.S. Pat. No. 5,198,193 describes a flow capillary device with multiple capillary paths leading towards a single reaction zone, each path having a different length and/or a valve to allow variation of timing of arrival of a liquid to the reaction zone. Such a device is ineffective as at each intersection of capillary paths including two different liquids, parallel flows are produced, analogous to the produced when a succeeding liquid is added onto an already wet capillary flow matrix.

EP 1044372 describes a lateral flow capillary device where sample and reagent liquids are added at two or more adjacent positions along a capillary flow matrix that is substantially a strip of bibulous material. Stripes of spacers made of impermeable hydrophobic material are placed perpendicularly to the flow direction to define liquid receiving zones and prevent mixing thereof. When liquids are added simultaneously an interface between two liquids is formed in the volume of the matrix underneath the spacer. Then, liquid from the first liquid zone migrates by capillary flow past the reaction zone to the liquid drain. After all the liquid in the first liquid zone is drained, the liquid in the second liquid zone migrates by capillary flow past the reaction zone to the liquid drain, and so forth. However, the structure of the device of EP 1044372 has many limitations: (i) the amount of liquid added to the liquid zones is limited; (ii) if the surface tension of the liquid is insufficient (e.g. due to size or detergents in the liquid, or due to insufficient capillary force) the liquid might spill from the lateral flow capillary device; (iii) the liquids must be added simultaneously, otherwise they will migrate from one liquid zone to a nearby liquid zone; and (iv) capillary paths might be formed in the space between a spacer and the capillary flow matrix through which two liquids in adjacent liquid zones may be mixed.

U.S. Pat. No. 4,981,786 describes a lateral flow capillary device with separate liquid reservoirs, which allow addition of two or more succeeding liquids without mutual contamination. Each reservoir is in capillary communication with capillary flow matrix leading to a reaction zone. However, the structure of the device might lead to liquid leaks away from the liquid zones through alternative capillary paths.

US 2013/0164193 describes a lateral flow capillary device comprising: (i) a unipath capillary flow matrix; (ii) a plurality of reservoirs each in fluid communication with the capillary flow matrix; and (iii) at least one pressure delivery system configured to apply uniform pressure thereon thereby urging capillary flow matrix. However, the device of US 2013/0164193 is extremely expensive, requires unique and relatively large permanent equipment and consumables, and utilizes pressure by using a set of springs to enable the sequential capillary flow of the liquid within the device, without an accurate pressure on the capillary flow matrix, leakages between the fluids reservoirs will occur which in turn will cause a mixing of the fluids from the reservoirs and incorrect sequential flow of the fluids. Moreover, the permanent device needs to be cleaned after each use in order to eliminate cross contaminations.

WO 2015/131142 describes and claims a lateral flow blotting assay, wherein the blotting device comprises an impermeable or hydrophobic barrier aimed at blocking the flow of a reagent from its reservoir into the lateral flow region.

Therefore, there is an unmet need for developing new and simple, single use sequential lateral flow capillary devices for the performance of multistep reactions hands-free for research and medical diagnosis purposes, which is also cost efficient, does not require electricity, and further enables preforming multi-analysis reactions simultaneously, while ensuring no mixing of the fluids in the reservoirs occurs to guaranty a sequential lateral flow through the structured flow matrix.

### SUMMARY OF INVENTION

It has now been found, in accordance with the present invention, that the drawbacks of known lateral flow capillary devices can be overcome by unique design that allows for a, single use, power-free and pressure-free sequential capillary flow even in a multistep reaction without the risk of spillage, leaks and inadvertent mixing between the different fluids in the various chambers of the device, while enabling a correct flow order of liquids from designated chambers within the device.

Accordingly, the present invention provides a disposable 3-D structured depended sequential lateral flow capillary device 100 comprising: (i) a housing having a proximal end and a distal end, said housing comprising: (a) a lower portion 110; (b) a middle portion 130; and (c) an upper portion 120, wherein all portions are coupled to one another, and defining an array of loadings cavities 101 and a drainage cavity 140, (ii) a 3-D structured capillary flow matrix 200 comprising: (a) a zigzag or wavy shaped proximal end having a number of waves which is identical to the number of said loadings cavities 101 and designed to fit into said loading cavities 101; (b) a distal end residing in the space between said middle portion 130 and said upper portion 120; and (iii) an absorption portion 141 residing, e.g., within said drainage cavity 140, wherein said absorption portion 141 is associated with said structured capillary flow matrix 200, either via direct contact or via an independent bridging member, or alternatively, said absorption portion 141 may be part of said structured capillary flow matrix 200, wherein upon loading liquids in said loading cavities 101, said zigzag or wavy shape of the proximal end of said structured capillary flow matrix 200 enables capillary action and insures that the flow order of said liquids is in the correct sequential order starting from the proximal loading cavity to the distal loading cavity, for performing all the required workflows for the detection and analysis of samples in a single step inside said disposable sequential lateral flow capillary device 100.

In certain embodiments, the present invention provides a disposable sequential lateral flow capillary device 100 comprising a housing having a proximal end and a distal end, said housing comprising: (a) a lower portion 110; (b) a middle portion 130; and (c) an upper portion 120, wherein all portions are coupled to one another, and defining an array of loadings cavities 101 and a drainage cavity 140. In specific embodiments, the disposable sequential lateral flow capillary device 100 further comprises a structured capillary flow matrix 200.

The present invention further provides a method for determining the presence of an analyte in a sample, by using the sequential lateral flow capillary device 100 of any one of the preceding claims, said method comprising the steps of: (a) providing a medium onto which said analyte is adsorbed or anchored; (b) placing said medium inside said device 100, either between said middle portion 130 and said capillary flow matrix 200, or between said capillary flow matrix 200 and said upper portion 120, and closing said upper portion 120; and (c) loading desired buffers and/or reaction liquids into said loadings cavities 101, wherein the liquid loaded in all said loadings cavities 101, contacts the structured capillary flow matrix 200 substantially simultaneously, and the flow of said buffers and/or reaction liquids from said loadings cavities 101, through said structured capillary flow matrix 200, and towards said absorption portion 141, is initiated by the addition of the liquids into said loadings cavities, and is carried out in the order of the loadings cavities.

The present invention further provides a method for determining the presence of an analyte in a sample, by using the sequential lateral flow capillary device 100 of any one of claims 1-14, said method comprising the steps of: (a) providing a structured capillary flow matrix 200 onto which a binding moiety capable of binding said analyte is anchored; (b) if needed, placing said structured capillary flow matrix 200 inside said device 100; and (c) loading said sample and any additional desired buffers and/or reaction liquids into said loadings cavities 101, in the correct flowing order, wherein the liquid loaded in all said loadings cavities 101, contacts the structured capillary flow matrix 200 substantially simultaneously, and the flow of said sample, buffers and/or reaction liquids from said loadings cavities 101, through said structured capillary flow matrix 200, and towards said absorption portion 141, is initiated by the addition of the liquids into said loadings cavities 101, and is carried out in the order of the loadings cavities 101.

### BRIEF DESCRIPTION OF THE DRAWINGS

FIGS. 1A-1D illustrate one possible configuration of a sequential lateral flow capillary device according to the invention: FIG. 1A illustrates a 3-dimentional view of the lower part with the internal loading cavities/chambers distribution; FIGS. 1B and 1D illustrate 3-dimentional front views of two possible configurations of the assembled device; and FIG. 1C illustrates a 3-dimentional rear view of the assembled device.

FIGS. 2A-2B illustrate another possible configuration of a sequential lateral flow capillary device according to the invention having only 3 loading cavities: FIG. 2A illustrates a 3-dimentional view of the lower part with the internal loading cavities/chambers distribution; and FIG. 2B illustrates a 3-dimentional front view of the assembled device.

FIG. 3 illustrates an exploded view of the 3 components of the device according to the invention: the lower portion; the middle portion; and the upper portion.

FIG. 4 illustrates an assembled device according to the invention without the structured capillary flow matrix, enlarging certain embodiments of the inner configuration after assembly.

FIG. 5 illustrates an assembled device according to the invention with the structured capillary flow matrix, enlarging certain embodiments of the inner configuration after assembly.

FIG. 6 is a protein detection analysis using a sequential lateral flow capillary device according to the invention.

### DETAILED DESCRIPTION OF THE INVENTION

The present invention provides a sequential lateral flow capillary device 100 and methods of using same for performing effective and repeatable single- or multi-step reactions, e.g., for analyte detection, serological testing, protein and/or DNA/RNA identification, any molecular biding analysis, etc.

Specifically, in certain embodiments, the present invention provides a disposable 3-D structured depended sequential lateral flow capillary device 100 comprising: (i) a housing having a proximal end and a distal end, said housing comprising: (a) a lower portion 110; (b) a middle portion 130; and (c) an upper portion 120, wherein all portions are coupled to one another, and defining an array of loadings cavities 101 and a drainage cavity 140; (ii) a 3-D structured capillary flow matrix 200 comprising: (a) a zigzag or wavy shaped proximal end having a number of waves which is identical to the number of said loadings cavities 101 and designed to fit into said loading cavities 101; (b) a distal end residing in the space between said middle portion 130 and said upper portion 120; and (iii) an absorption portion 141 residing, e.g., within said drainage cavity 140, wherein said absorption portion 141 is associated with said structured capillary flow matrix 200, either via direct contact or via an independent bridging member, or alternatively, said absorption portion 141 may be part of said structured capillary flow matrix 200, wherein upon loading liquids in said loading cavities 101, said zigzag or wavy shape of the proximal end of said structured capillary flow matrix 200 enables capillary action and insures that the flow order of said liquids is in the correct sequential order starting from the proximal loading cavity to the distal loading cavity, for performing all the required workflows for the detection and analysis of samples in a single step inside said disposable sequential lateral flow capillary device 100.

The sequential lateral flow capillary device 100 according to the present invention may be fabricated in any length, widths and thickness, and can be in any shape and size, depending on the designated use and user desire. For instance, the sequential lateral flow capillary device 100 of the invention may have a square shape of about 10×10 cm; about 11×11 cm; about 12×12 cm; about 13×13 cm; about 15×15 cm; about 20×20 cm; about 25×25 cm; about 30×30 cm, etc. In specific embodiments, the sequential lateral flow capillary device 100 of the invention may have dimensions that are smaller than from 10×10. In certain embodiments, the sequential lateral flow capillary device 100 of the invention may have rounded corners; it may be rectangular with any length and width; it may have a thickness of about 0.9, 1, 1.1, 1.2, 1.3, 1.4, 1.5, or 2 cm; etc.

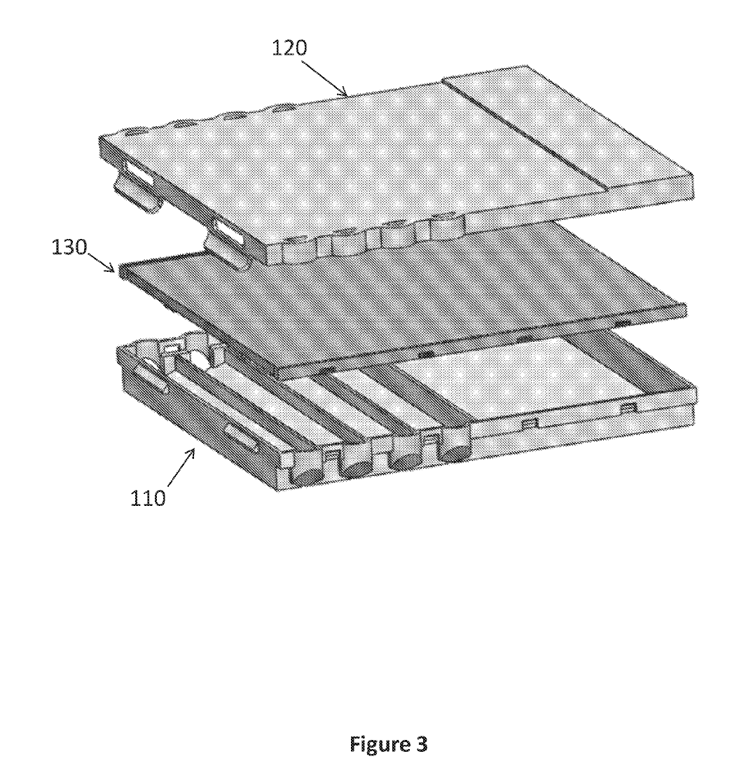

In specific embodiments, the present invention provides a disposable sequential lateral flow capillary device 100 comprising a housing having a proximal end and a distal end, said housing comprising: (a) a lower portion 110; (b) a middle portion 130; and (c) an upper portion 120, wherein all portions are coupled to one another, and defining an array of loadings cavities 101 and a drainage cavity 140 (FIG. 3).

In certain embodiments, the number of loading cavities 101 is 1, 2, 3, 4, 5, 6, 7, 8, 9, or 10. In other embodiments, the size and/or volume of each one of said loading cavities 101 is the same or different. In specific embodiments, some loading cavities 101 have the same size and/or volume while others have a different size and/or volume. For instance, the volume may range from about 3 to about 10 cm³; from about 4 to about 10 cm³; from about 5 to about 10 cm³; from about 6 to about 10 cm³; from about 7 to about 10 cm³; from about 3 to about 9 cm³; from about 3 to about 8 cm³; from about 3 to about 7 cm³; from about 3 to about 6 cm³; from about 3 to about 5 cm³; or from about 4 to about 6 cm³.

In certain embodiments, the location of said loading cavities 101 may vary according to the user's need. For instance, as illustrated in the figures, said loading cavities 101 are located within said lower portion 110 and underneath said middle portion 130. Alternatively, said loading cavities 101 may be located above said middle portion 130. In another alternative, said loading cavities 101 may be located parallel and horizontal to said middle portion 130. In other embodiments, the location of the designated openings 122 for filling said loading cavities 101 may vary. For instance, as illustrated in the figures, there may an opening in each side of the upper portion 120 above each loading cavity 101. Alternatively, there may only a single opening 122 for each loading cavity—at any location across said upper portion 120, e.g. on the side, in the middle or anywhere in between.

In certain embodiments, the lower portion 110 and the middle portion 130 of the sequential lateral flow capillary device 100 of the invention are joined together via suitable clip-on(s) or latching mean(s), optionally irreversibly. In other embodiments, the lower portion 110 and the upper portion 120 of the sequential lateral flow capillary device 100 of the invention are reversibly engageable via suitable clip-on(s) or latching mean(s) 123,124, thus enabling opening and closing the device, e.g., for placing-in or extracting-from a medium onto which an analyte or a binding moiety capable of binding a desired analyte is adsorbed or anchored.

In certain embodiments, the upper portion 120 of the sequential lateral flow capillary device 100 further comprises vertical and/or horizontal strengthening ribs 121, designed to prevent unintentional breakage when opened or closed. In other embodiments, such strengthening ribs may be also in the middle portion 130 and the lower portion 110. In certain embodiments, said strengthening ribs may be visible and external, or unseen and embedded within each portion.

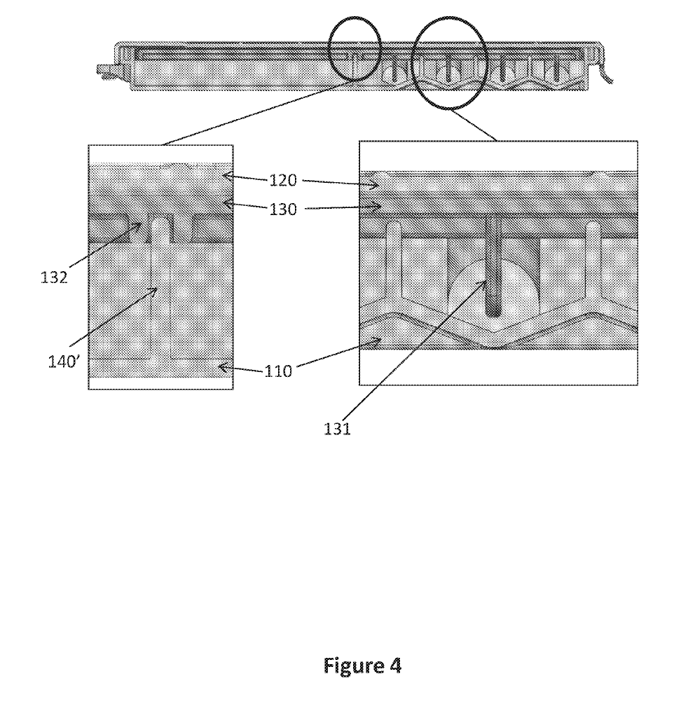

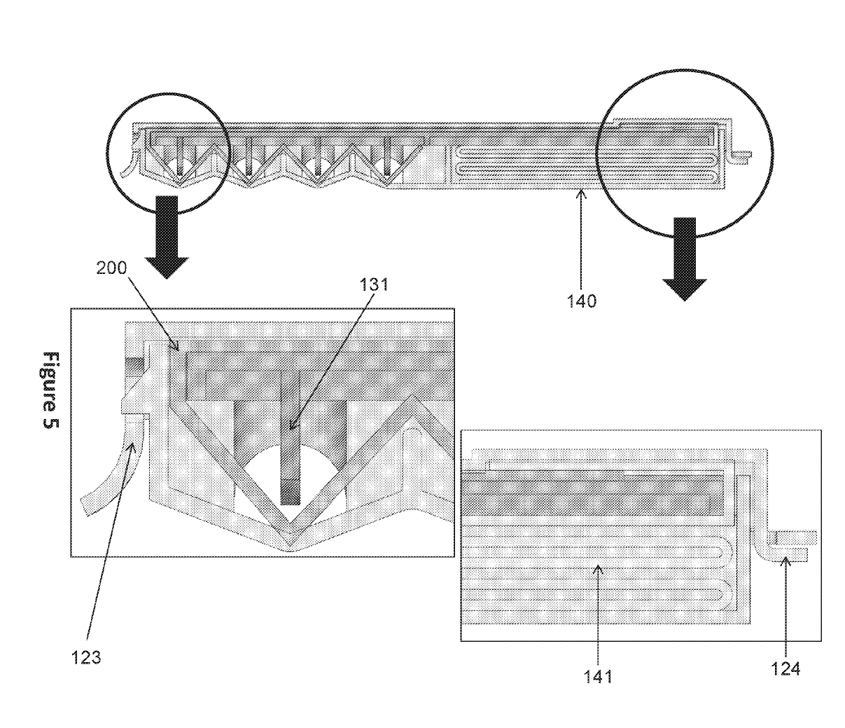

In certain embodiments, the middle portion 130 further comprises at least one vertical extension 131 extending from said middle portion 130 towards the bottom of each of said loadings cavities 101 (FIG. 4). The number and distribution of said vertical extensions 131 is according to the number and location of the loading cavities 101 in the lower section 110. In addition, the length of said vertical extensions 131 is according to the thickness and depth of the loading cavities 101, i.e. designed to reach almost to the bottom of each loading cavity without interfering with closing/attaching said middle portion 130 to said lower portion 110 (FIG. 4). These vertical extensions 131 are designed to push the structured portion of a structured capillary flow matrix 200 placed within the device 100 towards the bottom of each loading cavity 101 to enable adsorption of all liquids loaded therein (see FIG. 5).

In certain embodiments, the middle portion 130 of the sequential lateral flow capillary device 100 of the invention further comprises a rail or groove 132 designed to create a sealed barrier with a wall 140′ (FIG. 4) in the lower portion 110 located between said distal loadings cavity 101 and said drainage cavity 140. In specific embodiments, said rail or groove 132 further comprises sealing material, such as silicon, rubber or glue, for creating better sealing. Optionally, said rail or groove 132 is irreversibly connected to the said wall 140′ between said distal loadings cavity 101 and said drainage cavity 140, e.g., by glue, silicon or welding. Accordingly, once a structured capillary flow matrix 200 is placed within the device 100, said middle portion 130 is connected, optionally irreversibly, to the lower portion 110, and prevents movement of the structured capillary flow matrix 200 and passage of fluids from one loading cavity to the other, and from the loading cavities 101 to the drainage cavity 140 and the absorption portion 141 therein.

The three portions (110,120,130) of the sequential lateral flow capillary device 100 of the invention may be made of any substantially non-compliant material, such as metal, thermoplastic or organic polymer, plastic, polycarbonate, etc. They can be made of either transparent material or not or a combination thereof. A variety of such materials is well known in the art and may be used in the practice of the present invention without limitation and without departing from the spirit and scope thereof. Exemplary materials that may be used in the fabrication of a rigid plate include metals such as aluminum, stainless steel, chrome and the like, rigid thermoplastics such as polyether ether ketone (PEEK), polyetherketoneketone (PEKK), polysulfone, and the like. It is well within the skill level of the practitioner having ordinary skill level in the art to test a variety of materials for use in the present invention without undue experimentation.

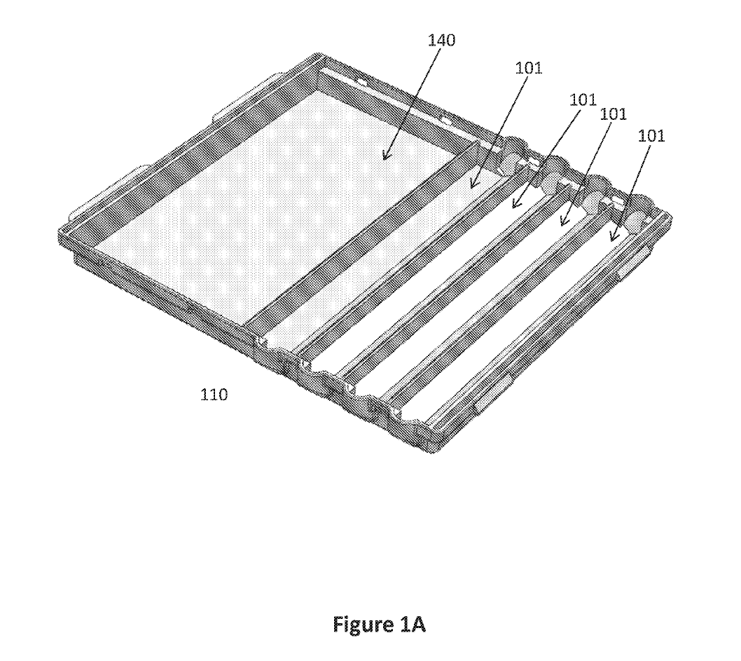

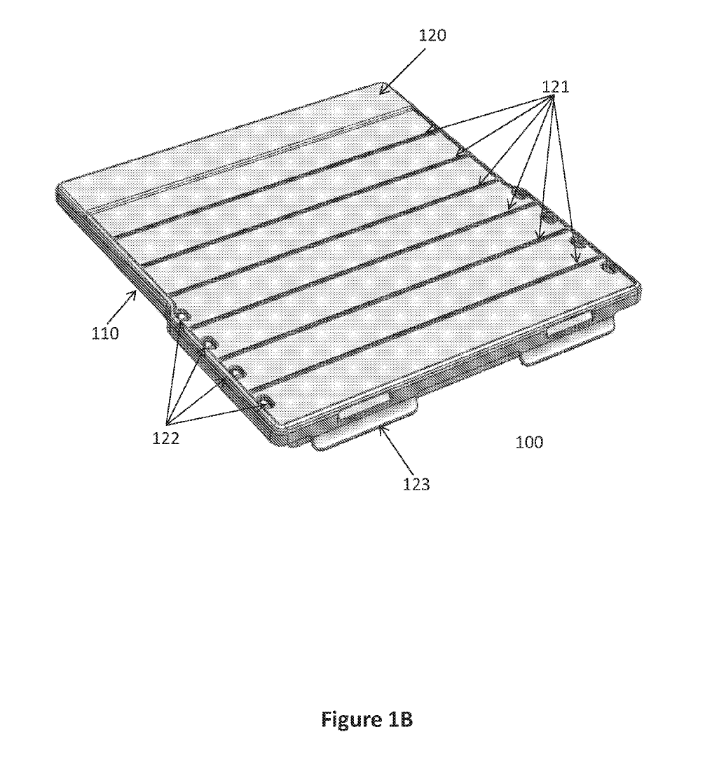

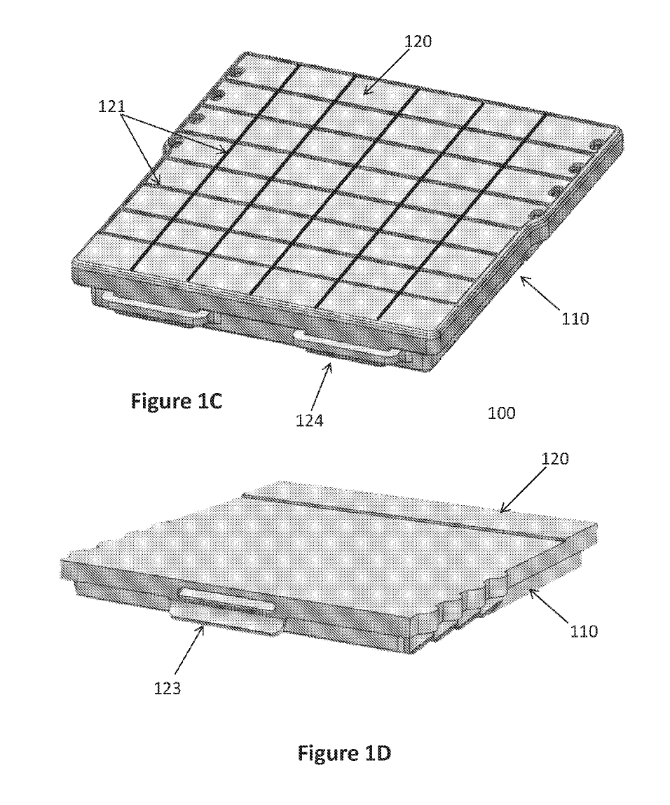

FIGS. 1-3 present exemplary embodiments of sequential lateral flow capillary devices 100 according to the invention are shown. Sequential lateral flow capillary device 100 may include a housing comprising a lower-, middle- and upper-portions (110,130,120, respectively). All portions are preferably made of a substantially rigid material, such as injection molded plastic. In some embodiments, the upper portion 120 may optionally include a clear/transparent window section so that a user may observe the sequential flow of liquids in each of the reservoirs into the structured capillary flow matrix. The lower portion 110 may further include protrusions and/or projections, optionally adjustable, to enable leveling the device 100 on any platform, such as a desk. In addition, the upper portion 120 may include grooves or rails to embrace said protrusions and/or projections thereby enabling easily mounting several devices one on the other in a single tower. Optionally, the middle portion 130 is attached to the lower portion 110 in an irreversibly manner, e.g. by welding, glue, or designated protrusions and grooves. In addition, latching means 123,124, disposed at both ends of the lower- and upper-portions, and enable reversibly engaging one to the other.

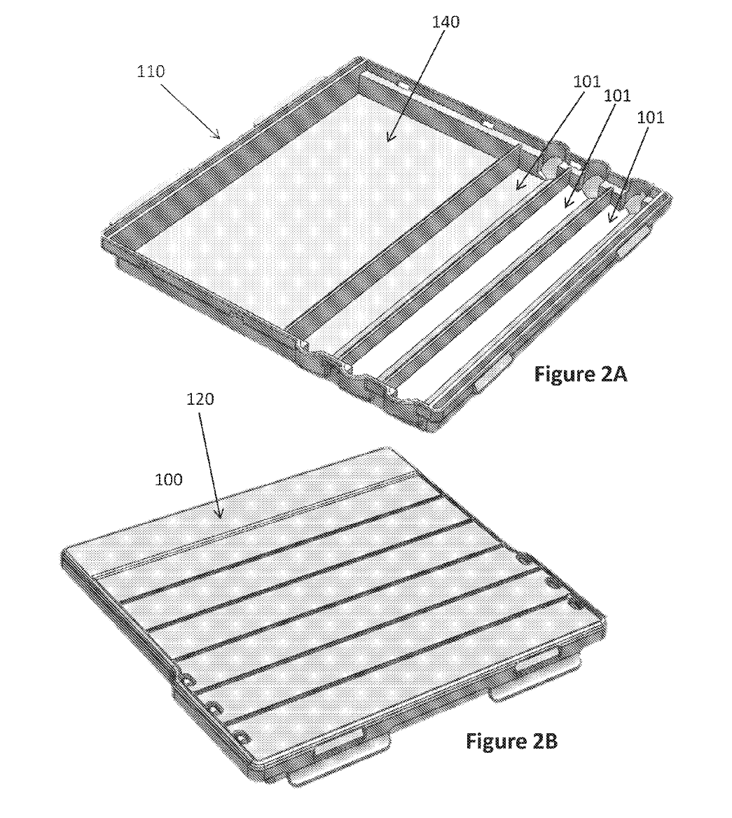

FIGS. 1B, 1C and 2B show the sequential lateral flow device 100 in an assembled configuration, where the loading cavities 101 are accessible to the user to add liquid/solution thereto via dedicated openings 122. It is to be understood that the loading cavities 101 may be sized to accept any volume of liquids, and all loading cavities may be either with the same volume or not, or combined (i.e. some cavities with the same volume, and others with a different volume). In certain embodiments of the present invention loading cavities 101 have a liquid volume in the range of about 1, 2, 3, 4, 5, 6, 7 ml to about 10, 15, 20, 25 or 30 ml; or from about 1, 3, 5 or 7 ml to about 7, 8, 9, 10, 12, 14, 15, 20, 25 or 30 ml; from about 3 ml to about 8 ml; or any other amount desired by the user.

In certain embodiments, the sequential lateral flow capillary device 100 of the invention further comprises a structured capillary flow matrix 200. In specific embodiments, said capillary flow matrix 200 is a structured capillary flow matrix 200, having, e.g., a zigzag or wavy shaped proximal end having a number of waves identical to the number of said loadings cavities 101 and designed so that each wave fits into a corresponding loading cavity. Moreover, any structured capillary flow matrix 200 which can keep the sequential flow of solutions from the loading wells to the structured capillary flow matrix 200 can be implemented. Thus, in another specific embodiment, each wave of the wavy shaped proximal end of said structured capillary flow matrix 200 resides within each cavity in said array of loadings cavities 101, and its distal end resides in the space between said middle portion 130 and said upper portion 120.

In certain embodiments, said structured capillary flow matrix 200 is bibulous, i.e. comprises a bibulous, porous or other cavity shaped material allowing capillary transport of liquids therethrough, i.e., the pores define a continuous system of capillary flow channels.

The term “bibulous material” includes, but is not limited to, materials composed of glass fiber paper or derivatized glass fiber paper, cellulose and its derivatives, nylons, PVDF, polysulfones, PTFE and polypropylene, paper and derivatized paper. Typically, the bibulous member consists of a series of fibers drawn together in parallel to form an open wick with some mechanical integrity due to bonding between the fibers, with the space between the fibers acting to form channels, which draw up liquid. Suitable fibers include polyester, polyamides such as nylons, and bi-component fibers such as polyethylene/polyester, nylon/polyester and the like. Bi-component polyethylene/polyester fibers typically comprise a polyester central core with an external sheath of polyethylene. Inherently hydrophobic fibers such as polypropylenes can also be used provided they are water wetable or, if necessary, are rendered water wetable by other components such as surfactants or hydrophilic polymers. In principle any wetable fiber is suitable.

In certain embodiments, the capillary matrices used according to the present invention can be of various forms including, but not limited to, sheets, columns, membranes, and compressed fibers. Suitable materials include but are not limited to porous materials and fibrous materials, including woven, rationally oriented and randomly oriented fibrous materials. Suitable materials include polymeric materials such as porous polymers including porous polyethylene, polypropylene, polytetrafluoroethylene (PTFE), ethylene vinyl acetate (EVA), polyether sulfone (PS), thermoplastic urethane (TPU), polyamide (e.g., Nylon 6) and copolymers thereof such as porous polymers manufactured by the Porex Corporation, Fairburn Ga., USA. Suitable materials include fibrous materials such as cellulose, cellulosic materials, cellulose derivatives, glass fibers, paper, filter paper, chromatographic paper, synthetic or modified naturally occurring polymers, such as nitrocellulose, cellulose acetate and cotton.

In specific embodiments, the structured capillary flow matrix 200 is attached to a substantially impermeable backing material, e.g. as known in the field of thin-layer chromatography where porous fibrous matter is bound to a solid impermeable backing. Suitable materials from which to form a backing include, but are not limited to, polyethylene, polypropylene, poly(4-methylbutene), polystyrene, polymethacrylate, poly(ethylene terephthalate), nylon, poly(vinyl butyrate), glass, ceramics, metals, polyurethane, neoprene, latex and silicone rubber.

A material exceptionally suitable for preparing the structured capillary flow matrix 200 of the invention is glass fiber especially plastic backed glass fiber, including glass fiber derivative such as glass fiber/cellulose/polyester matrices. Glass fiber membranes are relatively thick, (typically up to 2 mm), have pore sizes of 1-40 micron and a relatively high water flow rate (when compared to typical nitrocellulose matrix) allowing large sample and reagent flow through. An additional advantage of glass fiber, as noted above, is that glass fiber is relatively thick and soft.

In certain embodiments, the material used for preparing the structured capillary flow matrix 200 is nitrocellulose, e.g. plastic backed nitrocellulose, optionally having a pore size of between 0.45 and 15 micron. In another embodiment, the material used for preparing the structured capillary flow matrix 200 is porous polyethylene, e.g. having a pore size of between 0.2 and 20 micron.

The actual physical size of a structured capillary flow matrix 200 of the sequential lateral flow capillary device 100 of the present invention is determined by many factors especially the material from which the matrix is made and the specific use or uses for which the sequential lateral flow capillary device 100 is intended. That said, in some embodiments a sequential lateral flow capillary device 100 of the present invention is a manually operated lateral flow capillary device 100.

In certain embodiments, the structured capillary flow matrix 200 of the device 100 of the invention further comprises a distal absorption portion 141 (FIG. 5). In an alternative embodiment, said structured capillary flow matrix 200 is in contact with an absorption portion 141 located, e.g., within the drainage cavity 140 (FIG. 5), either directly or via a bridging member, said bridging member may be an independent unit, or part of the absorption portion 141 (as illustrated in FIG. 5).

In specific embodiments, upon loading liquids in said loading cavities 101 of the device 100 of the invention, said zigzag or wavy shape of the proximal end of said structured capillary flow matrix 200 enables capillary action and insures that the flow order of said liquids is in the correct sequential order starting from the proximal loading cavity to the distal loading cavity, for performing all the required workflows for the detection and analysis of samples in a single step inside said disposable sequential lateral flow capillary device 100.

The present invention provides a sequential lateral flow capillary device 100 and methods of using same. The device according to the invention allows performance of effective and repeatable multistep reactions for both research purposes as well as for diagnostic and medical tests, such as multistep specific binding assays.

Accordingly, in certain embodiments, the sequential lateral flow capillary device 100 of the invention can be used in diagnostic analytical methods for detecting an analyte, such as a biomarker, e.g. an antigen, antibody, metabolite, toxicant or other detectable material from human or other living source such as blood, urine, tissue, or from a non-living source such as an environmental source like water, soil or sewage. In certain embodiments, the analyte binds to an anti-analyte immobilized onto said capillary flow matrix 200, and then the bound analyte is detected directly or by a labeled reagent producing a detectable signal or that produces a detectable signal after being exposed to a third reagent. In other embodiments, the analyte is bound to a medium placed within the device 100 and the anti-analyte molecule or detector flows from the loading cavities 101 through said capillary flow matrix 200, and over the bound analyte, thus binding thereto and enabling detection by any suitable means.

Thus, in certain embodiments, the present invention provides a method for determining the presence of an analyte in a sample, by using the sequential lateral flow capillary device 100 of the invention, said method comprising the steps of: (a) providing a medium onto which said analyte is adsorbed or anchored, e.g. nitrocellulose or PVDF membranes; (b) placing said medium inside said device 100, either between said middle portion 130 and said structured capillary flow matrix 200, or between said structured capillary flow matrix 200 and said upper portion 120, and closing said upper portion 120; and (c) loading desired buffers and/or reaction liquids into said loadings cavities 101, wherein the liquid loaded in all said loadings cavities 101, contacts the structured capillary flow matrix 200 substantially simultaneously, and the flow of said buffers and/or reaction liquids from said loadings cavities 101, through said structured capillary flow matrix 200, and towards said absorption portion 141, e.g. located within said drainage cavity 140, is initiated by the addition of the liquids into said loadings cavities, and is carried out in the order of the loadings cavities, i.e. from the proximal loading cavity to the distal one.

In specific embodiments, the flow of liquid through said structured capillary flow matrix 200, from a distal loading cavity begins immediately after the liquid from the previous (proximal) loading cavity is depleted. This way, the device 100 of the invention enables passing liquids through said structured capillary flow matrix 200 in the correct and desired order in which they should flow. For example, a first fluid containing a first antibody is located in the proximal loading cavity and is the first to pass through said matrix 200; in the next loading cavity a washing buffer is loaded, which is the next in line to pass through said matrix 200; the following loading cavity contains a secondary antibody, which passes through the matrix only after the washing buffer from the previous loading cavity is depleted, and so forth, until all the liquids from all the loading cavities 101 are depleted.

In certain embodiments, two mediums having analytes attached thereon are placed inside the device 100 of the invention: one between the middle portion 130 and the structured capillary flow matrix 200, and the other between the structured capillary flow matrix 200 and the upper portion 120. This way it is possible to perform analysis and/or detection assays simultaneously on two separate mediums containing either the same samples (duplicates) or two different samples.

In other embodiments, the present invention provides a method for determining the presence of an analyte in a sample, by using the sequential lateral flow capillary device 100 of the invention, said method comprising the steps of: (a) providing a structured capillary flow matrix 200 onto which a binding moiety capable of binding said analyte is anchored; (b) if needed, placing said capillary flow matrix 200 inside said device 100; and (c) loading said sample and any additional desired buffers and/or reaction liquids into said loadings cavities 101, in the correct flowing order, wherein the liquid loaded in all said loadings cavities 101, contacts the structured capillary flow matrix 200 substantially simultaneously, and the flow of said sample, buffers and/or reaction liquids from said loadings cavities 101, through said capillary flow matrix 200, and towards said absorption portion 141, e.g. located in said drainage cavity 140, is initiated by the addition of the liquids into said loadings cavities 101, and is carried out in the order of the loadings cavities 101, i.e. from the proximal loading cavity to the distal one.

In certain embodiments, said analyte is a molecule, a protein, a virus, a bacteria or a cell. In other embodiments, said binding moiety is an antibody, a substrate or an inhibitor of an enzyme, a viral- or a bacterial-shell, a molecule having affinity to the analyte, or any other suitable moiety capable of binding the desired analyte. In specific embodiments, the analyte might be the binding moiety and vise-versa.

It is to be understood that the invention as described herein is not limited in its application to the details set forth herein. The invention can be implemented with other embodiments and can be practiced or carried out in various ways known to the skilled artisan in the art. It is also understood that the phraseology and terminology employed herein is for descriptive purpose and should not be regarded as limiting. The descriptions, materials, methods and examples are illustrative only and not intended to be limiting. Methods and materials similar or equivalent to those described herein can be used in the practice or testing of the present invention.

In certain embodiments, “analyte” refers to any material to be detected or quantitatively analyzed, such as, molecule, compound, composition, toxins, organic compounds, proteins, peptides, cells, microorganisms, bacteria, viruses, amino acids, nucleic acids, carbohydrates, enzymes, hormones, steroids, vitamins, drugs (including those administered for therapeutic purposes as well as those administered for illicit purposes), pollutants, pesticides, and metabolites of or antibodies to any of the above substances.

Generally, an analyte is found in a “sample” and the teachings of the present invention are applied to the sample to, e.g., determine the presence of or an amount of analyte present in a sample.

The term “sample” as used herein refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). In addition, a solid material suspected of containing the analyte can be used as the test sample once it is modified to form a liquid medium or to release the analyte. Pretreatment may involve preparing plasma from blood, diluting viscous fluids, and the like. Methods of treatment can involve filtration, distillation, separation, concentration, inactivation of interfering components, and the addition of reagents. Besides physiological fluids, other samples can be used such as water, food products, soil extracts, and the like for the performance of industrial, environmental, or food production assays as well as diagnostic assays. The selection and pretreatment of biological, industrial, and environmental samples prior to testing is well known in the art and need not be described further.

As stated above, the structured capillary flow matrix 200 may comprise a binding moiety attached thereto for the detection of an analyte in a sample. Thus, in certain embodiments, the present invention provides a sequential lateral flow capillary device 100 comprising a structured capillary flow matrix 200 that includes at least one reaction zone that comprises at least one binding or capturing moiety configured to capture at least one analyte flowing through the structured capillary flow matrix 200 in defined regions for conducting the assay reaction.

As explained above, the liquid from the loading cavities 101 flows through the structured/shaped capillary flow matrix 200 towards an absorption portion 141 residing, e.g. in a drainage cavity 140. An absorption portion is generally a component made of a bibulous material and having a liquid absorbing capacity that is significantly larger than that of a respective capillary flow matrix. Thus, in certain embodiments, the structured capillary flow matrix 200 is integrally formed with an absorption portion 141. Alternatively, the structured capillary flow matrix 200 does not comprise such an absorption portion 141 but is in direct or indirect contact therewith. Suitable materials from which an absorption portion can be made are described, e.g., in U.S. Pat. No. 4,632,901, such as, fibrous materials like cellulose acetate fibers, cellulose or cellulose derivatives, polyester, polyolefins, etc.

### EXAMPLES

### Possible Configurations of Sequential Lateral Flow Capillary Devices of the Invention

FIGS. 1 and 3 illustrate one embodiment of a sequential lateral flow capillary device 100 that includes four loading cavities 101: the first (proximal) three with the same volume and the last one (lateral) with a larger volume. The device further comprises a drainage cavity 140 for the absorption portion 141. Once the device 100 is closed, the user can fill the loading cavities 101 via designated openings 122 in the upper portion 120. The upper portion 120 may comprise strengthening ribs 121—either horizontal (FIG. 1B), vertical, or both (FIG. 1C). The upper portion 120 and the lower portion 110 may be detachable from one another and may be assembled via clip-ons or latching means located at the proximal and lateral ends 123,124. Alternatively, they can be connected via hinges at the distal end and latching means at the proximal end that enable opening and closing the upper portion 120.

FIG. 2 illustrates another embodiment of a sequential lateral flow capillary device 100 that includes three loading cavities 101: the first (proximal) two with the same volume and the last one (lateral) with a larger volume. The device further comprises a drainage cavity 140 for the absorption portion 141.

It should be understood that the number of loading cavities 101 can vary according to the user personal desire. In addition, not all loading cavities 101 need to be used by the end user, or may be filled with the same liquid in order to use larger volumes thereof.

### Possible Usages of the Sequential Lateral Flow Capillary Devices of the Invention

Using a Capillary Flow Matrix with an Integral Binding Moiety

For use, in accordance with the method of the present invention, a first amount of a first liquid, e.g. sample containing analyte placed in the 1^{st} (proximal) loading cavity, flows into a structured capillary flow matrix 200 through a reaction zone where said analyte binds to a binding moiety. A second amount of a second liquid, e.g. a labeled reagent, placed in the 2^{nd} loading cavity, flows into the structured capillary flow matrix 200 and pass said reaction area where it interacts and associates with the bound analyte. A third amount of a third liquid, e.g. a signal producing element, placed in the 3^{rd} loading cavity, flows into the structured capillary flow matrix 200 and reach the labeled reagent bound to the analyte, where it generates a visible/detectable reaction indicating the presence (or absence) of the analyte in the tested sample.

Although all liquids are loaded substantially together into the device 100, each liquid from each loading cavity flows according to the order of the loading cavities—first flows the liquid from the 1^{st} (proximal) loading cavity; the liquid from the next loading cavity flows only after the liquid from the first loading cavity has drained completely; and so forth. This assures correct order of fluid's flow and renders other actions or fluid replacements, redundant.

Thus, in certain embodiments of the invention, all the liquids are loaded substantially simultaneously to all the loading cavities 101 in the device. Alternatively, they can be loaded sequentially.

The above described method and device 100, contrary to the methods and lateral flow capillary devices of the prior art, allow performance of multistep reactions using a sequential lateral flow capillary device where each step is performed with a precise amount of reagent for a precise duration and in the correct order. Since leakage is not an issue since all the liquids are placed in confined and separate loading cavities 101, and since the duration of a reaction step is accurately determined by the volume of the different liquids added and the speed of capillary flow through the matrix 200, many different multistep experiments can be performed to yield repeatable results. Further, as the volume of liquid is the primary determinant of duration of a given step, the duration of a given step is easily modified if required, allowing performance of kinetic experiments.

Using a Capillary Flow Matrix with an Integral Binding Moiety and Other Reagents

In certain embodiments, the sequential lateral flow capillary device 100 is provided with a structured capillary flow matrix 200 comprising one or more reagents pre-loaded thereon. Such preloading of reagents is known in the art of lateral flow capillary devices, e.g. by drying reagents onto the matrix 200, by, e.g., freeze drying, spray drying, dispensing and air drying.

In such a device, a reagent passing through the structured capillary flow matrix 200 will interact with the pre-loaded reagent. In embodiments, at least one pre-loaded reagent is configured to react with an added analyte to produce a reaction product that is subsequently transported downstream along the structured capillary flow matrix 200 or identified on the spot.

### Manufacturing and Assembly of the Sequential Lateral Flow Capillary Device of the Invention

In general, manufacture and assembly of a sequential lateral flow capillary device 100 of the present invention is well within the ability of one skilled in the art upon perusal of the description and figures herein using any suitable method with which one skilled in the art is well acquainted. Suitable methods include methods that employ one or more techniques including but not limited to welding, casting, embossing, etching, free-form manufacture, injection-molding, microetching, micromachining, microplating, molding, spin coating, lithography or photo-lithography.

In an aspect of the present invention, a device and a kit are provided allowing the simple and cheap preparation of a custom sequential lateral flow capillary device in accordance with the teachings of the present invention.

In certain embodiments, the device 100 is sold as a whole, namely the housing is assembled with the structured capillary flow matrix 200 incorporated therein. Alternatively, the housing may be sold separately from the structured capillary flow matrix 200 which can be inserted to the housing by the user. In a specific embodiment, the housing is sold disassembled, namely the lower-, middle- and upper potions (110,120,130) are provided to the user which then assembles the device 100. This approach may save both delivery costs and storage space. It may also enable easy replacement of a damaged part.

In certain embodiments, for assembly the device 100 of the invention, an absorption portion 141 is placed in the drainage cavity 140; the proximal end of the structured capillary flow matrix 200 may be folded into a wavy or zigzag form, wherein the number of waves is identical to the number of loading cavities 101; then, each wave is placed in the appropriate loading cavity, and the middle part 130 is attached to the lower part 110, optionally in an irreversible manner, thereby closing over the absorption portion 141 in the drainage cavity 140 and over the proximal wavy part of the structured capillary flow matrix 200 in the loading cavities 101; next, the distal straight part of the structured capillary flow matrix 200 is folded over the top surface of the middle portion 130 and is brought into contact with the absorption portion 141, optionally via a bridging member; now the upper portion 120 is connected to the lower- and middle portions assembly. If the structured capillary flow matrix 200 comprises a reaction zone with an analyte binding moiety, the user may now simply add the tested sample and any other liquids and buffers into the loading cavities 101, wait for the reaction to occur, and analyze the results. Alternatively, the user may place one or two mediums with analytes bound thereon inside the device 100 and in contact with the structured capillary flow matrix 200, close the upper portion 120, place appropriate liquids and buffers in the loading cavities 101, and wait for the liquids to pass through the structured capillary flow matrix 200 and over said medium(s).

As is clear to one skilled in the art upon perusal of the description a lateral flow capillary device, including a sequential lateral flow capillary device of the present invention is easily custom built and modified with the use of embodiments of devices of the present invention and embodiments of kits of the present invention. For example, application of desired reagents to define a reaction zone or to preload a reagent onto a capillary matrix is simple to achieve.

### Detection of GAPDH in Whole Cell Lysate

Different concentrations of whole cell lysate were separated on an SDS-page gel and transblotted to a nitrocellulose membrane. The nitrocellulose membrane was blocked with 5% milk powder in TBST (TBS buffer+0.5% Tween 20) for 60 min.

The nitrocellulose membrane was then placed in a sequential lateral flow capillary device 100 of the invention between the middle portion 130 and the structured flow capillary matrix 200.

After closure of the upper portion 120, the following liquids/buffers were loaded into the loading cavities 101: (i) rabbit anti-human GAPDH in TBST into the 1^{st}, proximal, loading cavity; (ii) washing buffer (TBST) into the 2^{nd} loading cavity; (iii) goat anti-rabbit IgG in TBST into the 3^{rd} loading cavity; and (iv) washing buffer (TBST) into the 4^{th} loading cavity. Sequential draining of the 1^{st}, 2^{nd}, 3^{rd} and 4^{th} loading cavities, in accordance with the teachings of the present invention, was observed.

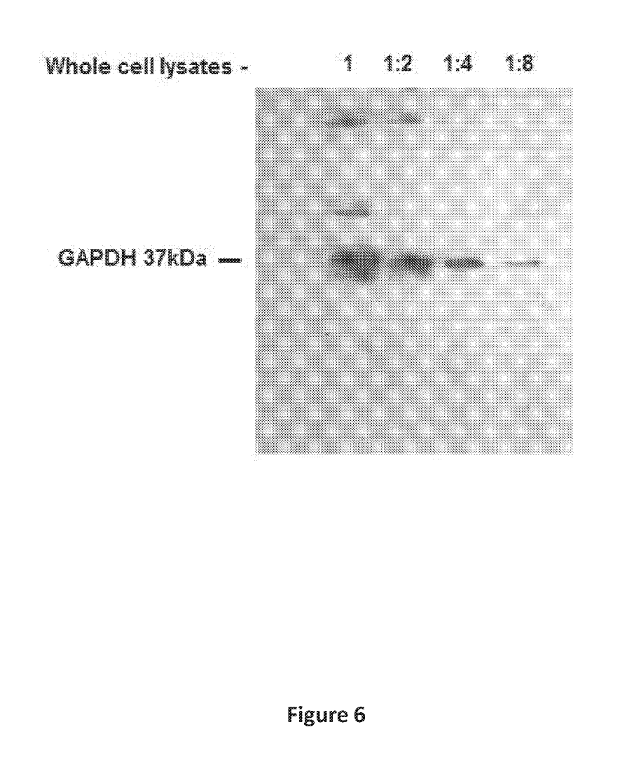

After about 2.5 to about 3 hours, the nitrocellulose membrane was removed from the device 100 and was subject to an ECL assay. The appearance of black lines (FIG. 6) at an intensity equivalent to the concentration of the loaded cell lysate, indicated the presence and amount of GAPDH in each sample.

Although the invention has been described in conjunction with specific embodiments thereof, it is evident that many alternatives, modifications and variations will be apparent to those skilled in the art. Accordingly, the present invention is intended to embrace all such alternatives, modifications and variations that fall within the spirit and broad scope of the appended claims. For example, the teachings of the present invention have been described where a reaction takes place at room temperature. In embodiments of the present invention, a sequential lateral flow capillary device is maintained in warmer or colder environment, for example a freezer, a refrigerator, or an incubator so that a reaction is performed at a temperature that is hotter or colder than room temperature or to ensure that a specific desired temperature is maintained. Embodiments in which a sequential lateral flow capillary device is maintained at a controlled temperature include during an entire reaction or during only part of a reaction.

1. A disposable 3-D structured depended sequential lateral flow capillary device comprising:
i) a housing having a proximal end and a distal end, said housing comprising:
(a) a lower portion;
(b) a middle portion; and
(c) an upper portion,
wherein all portions are coupled to one another, and defining an array of loadings cavities and a drainage cavity.

2. The device of claim 1, further comprising:
ii) a 3-D structured capillary flow matrix comprising:
c) a zigzag or wavy shaped proximal end having a number of waves which is identical to the number of said loadings cavities and designed to fit into said loading cavities;
d) a distal end residing in the space between said middle portion and said upper portion; and
iii) an absorption portion residing, e.g., within said drainage cavity,
wherein said absorption portion is associated with said structured capillary flow matrix, either via direct contact or via an independent bridging member, or alternatively, said absorption portion may be part of said structured capillary flow matrix,
wherein upon loading liquids in said loading cavities, said zigzag or wavy shape of the proximal end of said structured capillary flow matrix enables capillary action and insures that the flow order of said liquids is in the correct sequential order starting from the proximal loading cavity to the distal loading cavity, for performing all the required workflows for the detection and analysis of samples in a single step inside said disposable sequential lateral flow capillary device.

3. The device of claim 1, wherein said lower portion and said middle portion are joined together via suitable clip-on(s) or latching mean(s), optionally irreversibly.

4. The device of claim 1, wherein said lower portion and said upper portion are reversibly engageable via suitable clip-on(s) or latching mean(s).

5. The device of claim 1, wherein said upper portion further comprises vertical and/or horizontal strengthening ribs.

6. The device of claim 1, wherein said middle portion further comprises at least one vertical extension extending from said middle portion towards the bottom of each of said loadings cavities.

7. The device of claim 1, wherein said middle portion further comprises a rail or groove designed to create a sealed barrier with a wall located between said distal loadings cavity and said drainage cavity.

8. The device of claim 7, wherein said rail or groove further comprises sealing material, such as silicon, rubber or glue, and said rail or groove is irreversibly connected to the wall between said distal loadings cavity and said drainage cavity, e.g., by glue.

9. The device of claim 2, further comprising a structured capillary flow matrix having, e.g., a zigzag or wavy shaped proximal end with a number of waves which is identical to the number of said loadings cavities and designed to fit into said loading cavities.

10. The device of claim 9, wherein each wave of the wavy shaped proximal end of said capillary flow matrix resides within each cavity in said array of loadings cavities, and its distal end resides in the space between said middle portion and said upper portion.

11. The device of claim 9, wherein said structured capillary flow matrix further comprises a distal absorption portion.

12. The device of claim 9, wherein said structured capillary flow matrix is in contact with an absorption portion located, e.g., within the drainage cavity, either directly or via a bridging member.

13. The device of claim 11, wherein upon loading liquids in said loading cavities, said zigzag or wavy shape of the proximal end of said structured capillary flow matrix enables capillary action and insures that the flow order of said liquids is in the correct sequential order starting from the proximal loading cavity to the distal loading cavity, for performing all the required workflows for the detection and analysis of samples in a single step inside said disposable sequential lateral flow capillary device.

14. A method for determining the presence of an analyte in a sample, by using the sequential lateral flow capillary device of claim 1, said method comprising the steps of:
a) providing a medium onto which said analyte is adsorbed or anchored;
b) placing said medium inside said device, either between said middle portion and said capillary flow matrix, or between said capillary flow matrix and said upper portion, and closing said upper portion; and
c) loading desired buffers and/or reaction liquids into said loadings cavities,
wherein the liquid loaded in all said loadings cavities, contacts the structured capillary flow matrix substantially simultaneously, and the flow of said buffers and/or reaction liquids from said loadings cavities, through said capillary flow matrix, and towards said absorption portion, is initiated by the addition of the liquids into said loadings cavities, and is carried out in the order of the loadings cavities.

15. The method of claim 14, wherein the flow of liquids through said structured capillary flow matrix, from one (distal) loading cavity begins immediately after the liquid from the previous (proximal) loading cavity is depleted.

16. The method of claim 14, wherein two mediums are placed inside said device: one between said middle portion and said capillary flow matrix, and the other between said capillary flow matrix and said upper portion.

17. A method for determining the presence of an analyte in a sample, by using the sequential lateral flow capillary device of claim 1, said method comprising the steps of:
a) providing a structured capillary flow matrix onto which a binding moiety capable of binding said analyte is anchored;
b) if needed, placing said capillary flow matrix inside said device; and
c) loading said sample and any additional desired buffers and/or reaction liquids into said loadings cavities, in the correct flowing order,
wherein the liquid loaded in all said loadings cavities, contacts the structured capillary flow matrix substantially simultaneously, and the flow of said sample, buffers and/or reaction liquids from said loadings cavities, through said capillary flow matrix, and towards said absorption portion, is initiated by the addition of the liquids into said loadings cavities, and is carried out in the order of the loadings cavities.

18. The method of claim 17, wherein said analyte is a molecule, a protein, a virus, a bacteria or a cell.

19. The method of claim 17, wherein said binding moiety is an antibody, a substrate, an inhibitor, or a viral- or bacterial-shell.

20. The device of claim 12, wherein upon loading liquids in said loading cavities, said zigzag or wavy shape of the proximal end of said structured capillary flow matrix enables capillary action and insures that the flow order of said liquids is in the correct sequential order starting from the proximal loading cavity to the distal loading cavity, for performing all the required workflows for the detection and analysis of samples in a single step inside said disposable sequential lateral flow capillary device.
